(19) United States
(12) Patent Application Publication
(10) Pub. No.: US 2023/0218279 A1
(43) Pub. Date: Jul. 13, 2023

(54) BIOPSY NEEDLE DEVICE

(71) Applicant: InLine-Med GmbH, Magdeburg (DE)

(72) Inventor: Juan Sebastian Sanchez Lopez, Magdeburg (DE)

(73) Assignee: InLine-Med GmbH, Magdeburg (DE)

(21) Appl. No.: 18/010,375

(22) PCT Filed: Jun. 16, 2021
(86) PCT No.: PCT/EP2021/066194

§ 371 Date: Dec. 14, 2022

(30) Foreign Application Priority Data
Jun. 16, 2020 (DE) 10 2020 115 846.7

(51) Int. Cl. A61B 10/02

(52) U.S. Cl. CPC A61B 10/0275; A61B 2010/0208; A61B 2017/00438

(57) ABSTRACT

The invention relates to a biopsy needle device comprising at least one hollow outer needle, an inner needle longitudinally movably guided in the outer needle, and a housing. Such a biopsy needle device can be used to carry out a biopsy on a living being, offering improved ergonomics.

### FIELD OF THE INVENTION

The invention relates to a biopsy needle device having at least one hollow outer needle, an inner needle guided in a longitudinally movable manner in the outer needle, and a housing. Such a biopsy needle device can be used to carry out a biopsy on a living being.

### BACKGROUND OF THE INVENTION

Such a biopsy needle device is known, for example, from DE 198 48 513 A1.

### SUMMARY OF THE INVENTION

The invention is based on the object of providing a biopsy needle device which is improved further.

In one embodiment of the invention, the biopsy needle device has at least one manually actuable triggering element for triggering a relative movement between the outer needle and the inner needle. Such a triggering element enables the biopsy needle device to be operated by the user. The biopsy needle device can be configured here as a purely manual biopsy needle device or as a semiautomatic biopsy needle device. In the case of the pure manual embodiment, the user has to carry out the complete relative movement between the outer needle and the inner needle, as is required for carrying out the biopsy, by themselves manually using the manually actuable triggering element. In the case of the semiautomatic embodiment, the user only has to trigger a pretensioned movement mechanism by means of the triggering element and only has to carry out a comparatively small movement themselves. The further required relative movement between the outer needle and the inner needle for carrying out the biopsy is then carried out further automatically by the movement mechanism, e.g. by a pretensioned triggering spring.

The triggering element can accordingly serve in particular for triggering a semiautomatic relative movement between the outer needle and the inner needle.

In one embodiment of the invention, the biopsy needle device has at least one finger grip element for holding and/or for operating the biopsy needle device by means of at least one finger of the user. By this means, the manual operation of the biopsy needle device is configured to be simpler and more pleasant and thus, as a result, the ergonomics are improved. The finger grip element can be configured in particular as a finger grip element which can receive just one finger of a user or which can be operated just by one finger of the user. For example, the finger grip element can be annular with an inner recess which is of a size such that it can receive a finger of a user, but is not suitable for receiving several fingers.

In this connection, it should be mentioned that the biopsy needle device can also be gripped or held on its housing by the user. However, the housing itself is not considered to be a finger grip element within the meaning of the present application. The housing can have, e.g. laterally, a notch or indentation which serves as the finger grip element.

It is advantageous if the inner needle is connected immovably in the longitudinal direction to the triggering element. The triggering element can be, for example, part of a displacement element of the biopsy needle device. The outer needle can be fastened immovably in the longitudinal direction to an outer needle holder.

In one embodiment of the invention, the biopsy needle device has only one finger grip element. The one finger grip element can be present here in addition to the triggering element and, for example, can be arranged spaced apart from the triggering element. In this way, the biopsy needle device can be configured to be very compact and small, and therefore it can be used with little space being required. The one finger grip element can serve here, for example, for receiving the middle finger of the user. In this case, the triggering element can be actuated, for example, by the index finger. Alternatively, the triggering element can be actuated by means of the thumb of the user, which permits particularly precise holding of the biopsy needle device.

In one embodiment of the invention, the biopsy needle device has one or more finger grip elements which are arranged asymmetrically with respect to a longitudinal axis of the outer needle. This also makes it possible to improve the operability and ergonomics of the biopsy needle device. In addition, particularly compact designs of the biopsy needle device are also possible as a result. Here too, a triggering element can be present in addition to the finger grip elements, or the triggering element can be formed by one or more finger grip elements.

In one embodiment of the invention, the biopsy needle device has at least one finger grip element at the proximal end of the housing. The proximal end of the housing is the end facing the user and accordingly that end of the housing which faces away from the patient when the biopsy needle device is used on the patient in order to carry out a biopsy. Accordingly, the distal end of the housing is the end facing the patient. Such a finger grip element can be used to realize completely novel operating techniques of the biopsy needle device for the user, for example a two finger or three finger operating technique, in which, without changing the gripping position of the fingers of the user on the biopsy needle device, both an initial initialization movement, in which a position present between inner needle and outer needle before carrying out the biopsy is set, and also the triggering movement for carrying out the biopsy can be carried out. In this way, the biopsy needle device is operatable completely single-handedly.

In one embodiment of the invention, the biopsy needle device has precisely three finger grip elements. The operability of the biopsy needle device can also be further improved as a result. In particular, a single-handed operability using a three-finger operating technique both during the initial initialization movement and in the triggering movement for carrying out the biopsy is assisted.

In the case of a semiautomatic biopsy needle device, the initialization movement serves for pretensioning the movement mechanism or the triggering spring thereof.

In one embodiment of the invention, the biopsy needle device has at least one pretensioning operating element which can be used to carry out a relative movement between the outer needle and the inner needle in a tensioning direction counter to the force of a triggering spring of the biopsy needle device. The triggering spring can be a tension spring or a compression spring depending on the design of the movement mechanism. The triggering spring can be configured, for example, as a spiral spring, torsion spring or other resilient element, e.g. as a block made of an elastic material. The triggering spring can be a single spring or an arrangement consisting of a plurality of springs.

In one embodiment of the invention, the biopsy needle device is configured in such a manner that at least one finger grip element or an arrangement consisting of at least two finger grip elements is mounted displaceably relative to the housing of the biopsy needle device in the direction of the longitudinal axis of the outer needle. In this way, the displaceable finger grip element or the displaceable arrangement consisting of at least two finger grip elements can be used for improved operation of the biopsy needle device, e.g. as a triggering element and/or as a pretensioning operating element.

With the displaceably mounted finger grip element or the displaceably mounted arrangement consisting of at least two finger grip elements, the biopsy needle device can be configured in particular in such a manner that the entire length does not increase when the triggering spring is pretensioned. This is possible as a result of the fact that the displaceably mounted finger grip element or the displaceably mounted arrangement consisting of at least two finger grip elements are only displaceable within the housing. Operating advantages for the user can also be realized as a result.

In one embodiment of the invention, the manually actuable triggering element is actuable in an actuating direction which is arranged transversely with respect to the longitudinal axis of the outer needle. In this way, the triggering movement can take place so to say laterally. This likewise permits different types of operation of the biopsy needle device. In addition, it is more simply possible to carry out a remote triggering of the triggering element by means of an external actuator, for example in order to avoid the user, i.e. the doctor, having to carry out the triggering within the range of x-ray radiation. The remote triggering of the triggering element means that the user does not have to hold their hand within the range of the x-ray radiation.

The embodiments of the biopsy needle device described previously and below can be combined with one another as desired.

In the previously explained embodiment with the lateral actuating option, i.e. the actuating direction arranged transversely with respect to the longitudinal axis of the outer needle, the biopsy needle device can be configured optionally with or without a finger grip element or finger grip elements. In particular in an embodiment which is specially configured for remote triggering by means of an external actuator, a finger grip element may be entirely dispensed with.

According to one embodiment of the invention, the biopsy needle device has a guide, e.g. in the housing of the biopsy needle device, in which guide the triggering element is guided. The guide defines a movement direction of the triggering element during the triggering operation transversely with respect to the longitudinal axis of the outer needle. For example, a right angle between the movement direction of the triggering element and the longitudinal axis of the outer needle can be defined by the guide.

In the case of the previously mentioned embodiments of the biopsy needle device, the latter can have at least one manually actuable triggering element for triggering a relative movement between the outer needle and the inner needle. Advantageously in this case, at least one finger grip element or an arrangement consisting of at least two finger grip elements can be configured simultaneously as such a triggering element. For example, the previously mentioned, displaceably mounted finger grip element or the displaceably mounted arrangement consisting of at least two finger grip elements can form such a triggering element. This likewise has an advantageous effect on single-handed use of the biopsy needle device. In addition, the biopsy needle device can be configured without a separate triggering element, as a result of which the design is simplified.

In the case of a semiautomatic biopsy needle device, the triggering element can serve simultaneously also as a pretensioning operating element, i.e. as a manual operating element for pretensioning the semiautomatic movement mechanism. In this way, the biopsy needle device is operable single-handedly. The triggering element serves for triggering a relative movement between the outer needle and the inner needle, said relative movement being brought about by the force of the pretensioned triggering spring and being opposed to the tensioning direction.

The inner needle can be configured as a solid needle, without an inner cavity. Advantageously, the inner needle, shortly before its distal end, can have a recess which serves as a tissue receiving pocket.

According to one embodiment of the invention, provision is made that the biopsy needle device has, in addition to the pretensioning operating element, a triggering element for triggering a relative movement between the outer needle and the inner needle, said relative movement being brought about by the force of the pretensioned triggering spring and being opposed to the tensioning direction. The pretensioning operating element makes it possible for a semiautomatic biopsy needle device to be brought into a pretensioned state in which it can then be triggered by the separate, manually actuable triggering element for automatically carrying out the further requirement relative movement between the outer needle and the inner needle to carry out the biopsy procedure. The triggering element can be arranged here in particular at a distance from the pretensioning operating element such that an unintended actuation does not take place. The biopsy needle device can also have more than one triggering element. The triggering elements can then be arranged at different points of the biopsy needle device and can be actuable, for example, in different actuating directions.

According to one embodiment of the invention, provision is made that the biopsy needle device has a holding mechanism which is used, when the triggering spring is pretensioned, to hold the pretensioning operating element, or a component connected thereto, in at least one holding position, in particular in at least two different holding positions, in relation to the force of the pretensioned triggering spring. By this means, single-handed operation of the biopsy needle device is simplified even further and configured more favorably for the user since the user does not have to apply any force in order to hold the pretensioned triggering spring. The holding mechanism can be configured, for example, as a latching mechanism.

According to one embodiment of the invention, provision is made that one finger grip element, a plurality of finger grip elements or all of the finger grip elements has/have a ring shape, the ring shape being able to be configured as a ring completely closed circumferentially or a ring which is open at one point. This permits secure receiving of one finger in one finger grip element each. The ring shape can be circular, oval or any other ergonomically suitable ring shape. If the biopsy needle device has a plurality of finger grip elements, these can be configured identically or differently. In addition, the finger grip element can be configured as a notch or indentation on the housing.

According to one embodiment of the invention, provision is made that one finger grip element, a plurality of finger grip elements or all of the finger grip elements is/are configured as a separate component which is coupled rigidly or movably to the housing. The production of the biopsy needle device is thereby simplified. In particular, comparatively simple injection molds can be used for producing the components.

According to one embodiment of the invention, provision is made that the just single finger grip element of the biopsy needle device is arranged on the housing in a manner laterally offset with respect to the longitudinal axis of the outer needle. This permits an ergonomically particularly favorable use of the biopsy needle device with just a single finger grip element. The finger grip element can be arranged in particular on the housing in a manner offset with respect to the longitudinal axis of the outer needle to an extent such that the longitudinal axis does not intercept the just one finger grip element.

According to one embodiment of the invention, provision is made that the distance between the finger grip element arranged at the proximal end of the housing and the distal end of the housing is unchangeable. This permits a particularly ergonomic configuration of the biopsy needle device for single-handed operation. In this way, at least the housing region of the biopsy needle device has a constant length which can be calculated by the user in advance and which also does not change during operation of the biopsy needle device. This is advantageous, for example, in the event of limited movement space of the biopsy needle device, e.g. in a CT tube. The finger grip element arranged at the proximal end of the housing can be connected immovably here to the housing. For example, the finger grip element arranged at the proximal end of the housing can protrude from the housing in a direction facing away from the distal end (close to the patient) of the housing.

According to one embodiment of the invention, provision is made that the distance between the finger grip element arranged at the proximal end of the housing and other finger grip elements of the biopsy needle device is at least 2 cm or at least 50% of the housing length of the biopsy needle device in every operating state of the biopsy needle device. The distance can also be at least 3 cm. The finger grip element arranged at the proximal end of the housing is therefore arranged ergonomically particularly pleasantly in comparison to the housing length. The housing length is considered here to be the distance between the distal and the proximal end of the housing. By this means, distances which are too small between the finger grip elements are avoided, which is also favorable for an ergonomic single-handed operation of the biopsy needle device.

According to one embodiment of the invention, provision is made that a docking holder is arranged on the housing of the biopsy needle device for holding an external actuator with which the triggering element can be actuated by the user by means of remote triggering. The external actuator means that the user of the biopsy needle device can be protected, for example in the event of x-ray examination, from holding their hand within the range of the x-ray radiation in order to actuate the triggering element. Remote triggering of the triggering element is therefore possible.

The docking holder can permit in particular a form-fitting coupling of the external actuator to the housing of the biopsy needle device (in the sense of “docking”).

The invention also relates to a set consisting of
a biopsy needle device having at least one hollow outer needle, an inner needle guided in a longitudinally movable manner in the outer needle, and a housing, wherein the biopsy needle device has at least one manually actuable triggering element for triggering a relative movement between the outer needle and the inner needle, and
an external actuator with which the triggering element of the biopsy needle device can be actuated by the user by means of remote triggering.

The previously explained advantages can also be achieved as a result. In particular, the previously mentioned advantageous remote triggering of the biopsy needle device can be carried out, and therefore the user can be protected against unnecessary radiation exposure during an x-ray examination or other investigations.

According to one advantageous embodiment of the invention, the external actuator has at least one fastening element with which the external actuator is fastenable to the biopsy needle device in a form-fitting and/or force-fitting manner. For example, the fastening element can be configured as a counterpart to the previously mentioned docking holder. However, the biopsy needle device can also be formed without a docking holder. For example, the fastening element of the external actuator can be configured in such a manner that it engages around or engages over a certain region of the housing of the biopsy needle device. In this way, the user can couple the external actuator to the biopsy needle device with little effort.

According to one advantageous embodiment of the invention, provision is made that the external actuator has an actuation plunger for remote actuation of the triggering element of the biopsy needle device. The actuation plunger is longitudinally movable in a direction which runs transversely with respect to the longitudinal axis of the outer needle. For example, the actuation plunger can be longitudinally movable in a direction which runs substantially perpendicularly to the longitudinal axis of the outer needle. In this way, the biopsy needle device can be triggered so to say from the side by the external actuator or the actuation plunger thereof. This permits a space-saving and ergonomic handling of the biopsy needle device with the external actuator fastened thereto, even in confined space conditions, e.g. in a CT tube.

It is conceivable for a pistol grip to be arranged at an end region of the external actuator. This has the advantage that the pistol grip constitutes an ergonomically adapted handle for the user, and therefore the force which is to be applied can be minimized and at the same time the engagement precision increased. Furthermore, improved grippiness and stability in guiding and orienting the biopsy needle device is achieved.

The invention will be explained in more detail below using drawings and by reference to exemplary embodiments:

### BRIEF DESCRIPTION OF THE DRAWING

FIG. 1 shows the components of a biopsy needle device in a first embodiment in an exploded illustration, and

FIG. 2 shows the biopsy needle device according to FIG. 1 in a side view, and

FIG. 3 shows a view of a detail of the biopsy needle device according to FIG. 2, and

FIG. 4 shows a perspective view of a displacement element, and

FIG. 5 shows a perspective view of an outer needle holder, and

FIG. 6 shows a side view of a housing part, and

FIG. 7 shows a front view of the housing part according to FIG. 6, and

FIG. 8 shows the housing part according to FIG. 6 in a side view from the opposite side, and

FIG. 9 shows a sectional view of the housing part according to FIG. 8 in the section plane A-A shown there, and

FIG. 10 shows a side view of a further housing part with a perspectively illustrated finger grip element, and

FIG. 11 shows a second embodiment of a biopsy needle device in an exploded illustration, and

FIG. 12 shows a third embodiment of a biopsy needle device in an exploded illustration, and

FIG. 13 shows the biopsy needle device according to FIG. 12 in a top view, and

FIG. 14 shows the biopsy needle device according to FIG. 12 in a side view, and

FIG. 15 shows the housing part of the biopsy needle device according to FIG. 12 and the displacement element thereof in a perspective illustration, and

FIG. 16 shows the outer needle holder of the biopsy needle device according to FIG. 12 in a perspective illustration, and

FIG. 17 shows a fourth embodiment of a biopsy needle device in an exploded illustration, and

FIG. 18 shows an embodiment of an external actuator in an exploded illustration, and

FIG. 19 shows a fifth embodiment of a biopsy needle device in an exploded illustration, and

FIG. 20 shows a biopsy needle device according to FIG. 19 in a sectioned side view.

### DETAILED DESCRIPTION OF THE INVENTION

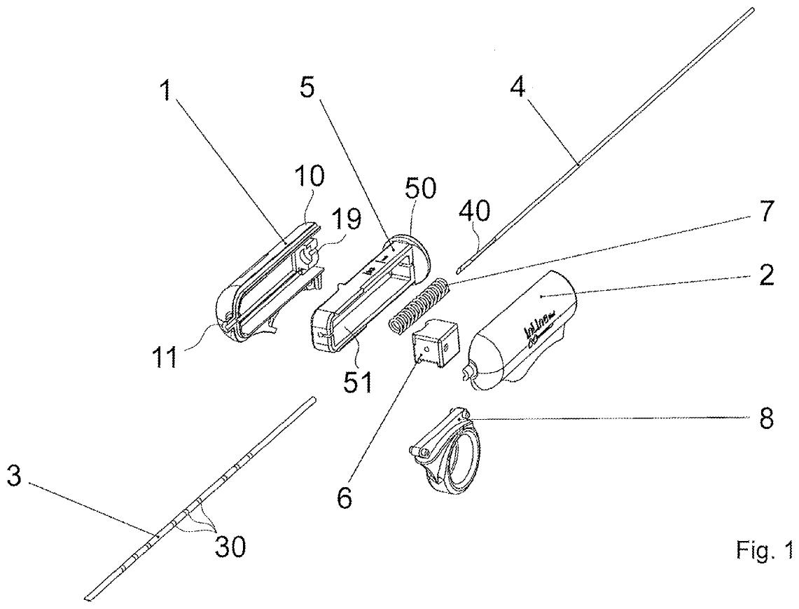

FIG. 1 shows a biopsy needle device with a housing 1, 2 which has two housing parts. The housing parts 1, 2 can be configured, for example, in the form of half shells. The biopsy needle device has just one finger grip element 8 which can be formed as a separate component and is fastened to the housing parts 1, 2. The finger grip element 8 can also be configured as a common component with the housing. In addition, the biopsy needle device has a hollow outer needle 3 and an inner needle 4 guided in a longitudinally movable manner in the outer needle 3. On the outer side of the outer needle 3 a penetration depth marking 30 is arranged, with reference to which the insertion depth of the outer needle 3 during a biopsy can be read. The inner needle 4 has a tissue window 40 for receiving the tissue sample in a biopsy.

As further parts of a semiautomatic movement mechanism, the biopsy needle device has an outer needle holder 6, a displacement element 5 and a triggering spring 7. In the assembled state, the outer needle holder 6 is arranged in an elongate interior space 51 of the displacement element 5. The displacement element 5 is arranged within the housing 1, 2 in the region in which the outer needle holder 6 is arranged in the interior space 51. The displacement element 5 protrudes out of the housing 1, 2 with a manual operating element which in this case is configured simultaneously as the triggering element 50 and is the pretensioning operating element. The triggering spring 7 is clamped between the outer needle holder 6 and a spring bearing 19, which serves as a counterbearing fixed on the housing. The spring bearing 19 can be formed on one or on both housing parts 1, 2. The outer needle 3 is fixedly connected at its proximal end to the outer needle holder 6, for example is adhesively bonded thereto. The inner needle 4 is guided through an inner cavity of the outer needle 3.

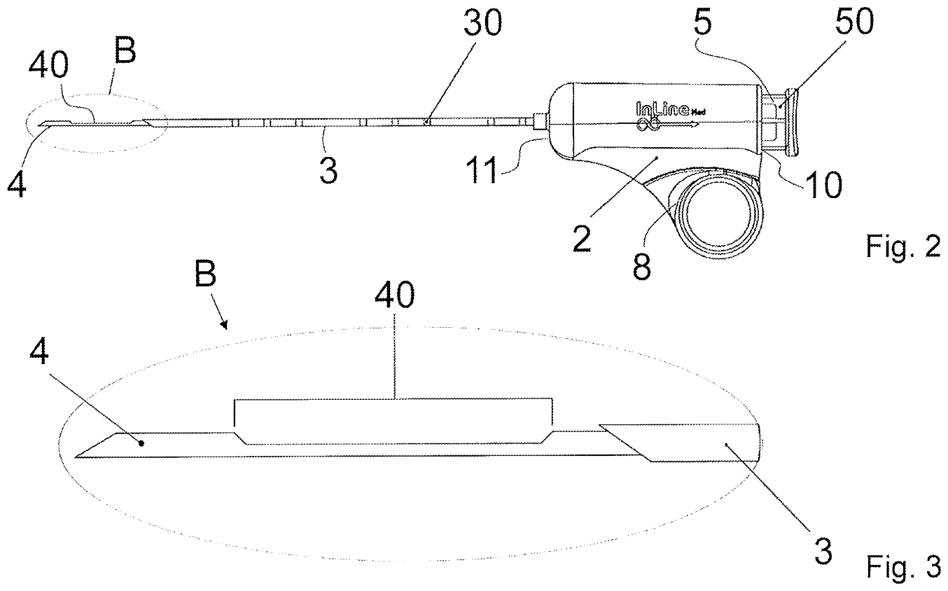

FIG. 2 shows the biopsy needle device according to FIG. 1 in the assembled state in a side view. It can be seen that the displacement element 5 with the triggering element 50 protrudes out of the housing 1, 2 at the proximal end 10 of the housing 1, 2.

In addition, the distal region of the biopsy needle device with the tissue window 40 formed on the inner needle 4 can be seen. Said distal region is additionally illustrated in enlarged form as detail enlargement B in FIG. 3. It is seen that the tissue window 40 is configured as an elongate recess, i.e. the cross-sectional area of the inner needle 4 is reduced in this region.

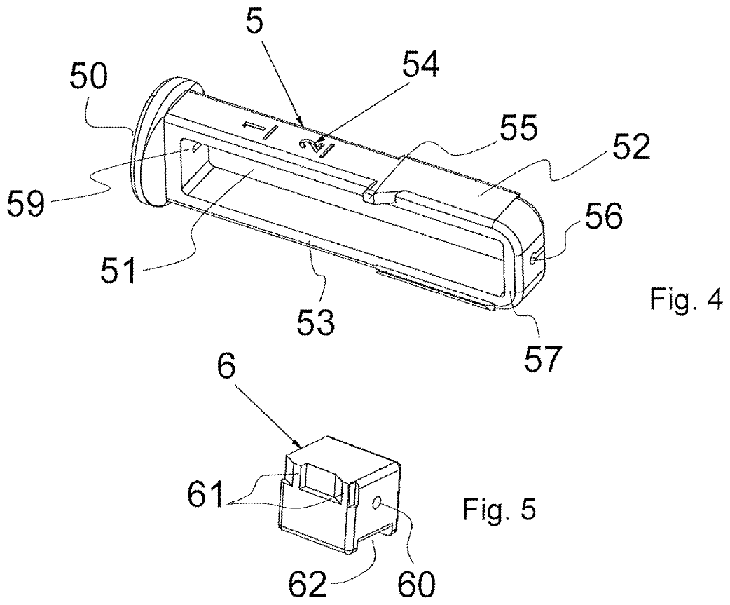

FIG. 4 shows the displacement element 5 in an enlarged detailed view. It can be seen that the displacement element 5 has two longitudinal struts 52, 53 spaced apart from each other. At the proximal end, the longitudinal struts 52, 54 lead into the manual triggering element 50. At the distal end, the longitudinal struts 52, 53 lead into a transverse strut 57. In the transverse strut 57 there is a through hole which forms an outer needle guide 56 through which the outer needle 3 is guided displaceably. At the triggering element 50 or in the vicinity of the triggering element 50, there can be an opening, which serves as an inner needle receptacle 59, on the displacement element 5. The inner needle 4 can be fastened to said inner needle receptacle 59.

The displacement element 5 serves as a tensioning device for tensioning the semiautomatic movement mechanism of the biopsy needle device. For the tensioning, the triggering element 50 is gripped by the user and pulled out of the housing 1, 2 in the proximal direction, as a result of which parts of the longitudinal struts 52, 53 emerge from the housing 1, 2 and are visible. The user can observe the cutting length markings 54 provided on the displacement element 5, e.g. on a longitudinal strut 52, and can select one of a plurality of pretensioning positions, e.g. from two different pretensioning positions, on the basis of said cutting length markings 54. During this pretensioning movement, the outer needle holder 6 arranged in the interior space 51 is carried along by the transverse strut 57 and, in the process, pressed against the triggering spring 7 mounted on the spring bearing 19 fixed on the housing. The triggering spring 7 is compressed in this way and pretensioned to a relatively great extent.

In addition, a latching release element 55 can be arranged on the displacement element 5, e.g. on a longitudinal strut 53. By means of the latching release element 55, a latching of the outer needle holder 6 which took place previously during the tensioning can be released in the event of a triggering movement of the semiautomatic movement mechanism.

As mentioned, the manual operating element of the displacement element 5 serves, during the tensioning, as the pretensioning operating element and then, during the triggering, as the manually actuable triggering element 50.

FIG. 5 shows the outer needle holder 6 in an enlarged illustration. The outer needle holder 6 is guided in the housing 1, 2 in linear guide elements. For this purpose, the outer needle holder 6 has a dedicated guide element 62 which is guided in at least one linear guide element of the housing 1, 2. Furthermore, the outer needle holder 6 has a receiving opening which forms an outer needle receptacle 60 for receiving and fastening the outer needle 3. In addition, at least one first latching element 61 is arranged on the outer needle holder 6. In the exemplary embodiment illustrated, there are two first latching elements 61 which correspond to two different latching positions during the pretensioning movement in accordance with the cutting length markings 54.

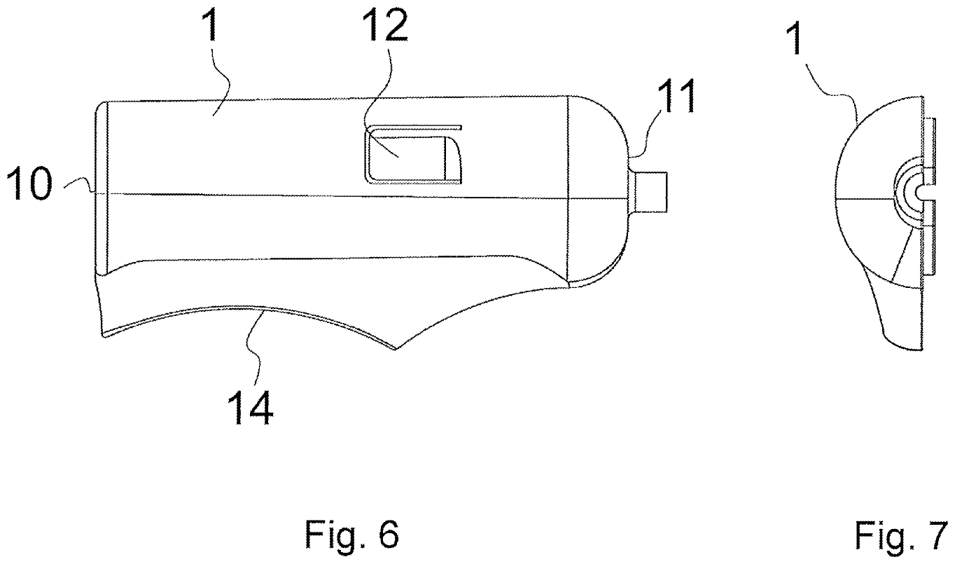

FIG. 6 shows the first housing part 1 in a side view. In the lower region there is an attachment point 14 at which the finger grip element 8 is added onto the housing parts 1, 2. In addition, on the housing part 1 there is a second latching element 12, e.g. in the form of an elastically deflectable latching tab, which can be realized, for example, by a U-shaped cutout being introduced on the housing part 1, in which cutout the latching tab is formed as remaining material. The second latching element 12 interacts with the first latching elements 61 of the outer needle holder 6 in order to fix the outer needle holder 6 in the desired latching positions.

FIG. 7 shows, for clarification, the first housing part 1 in a front view, i.e. in a top view of the distal end of the housing 1, 2.

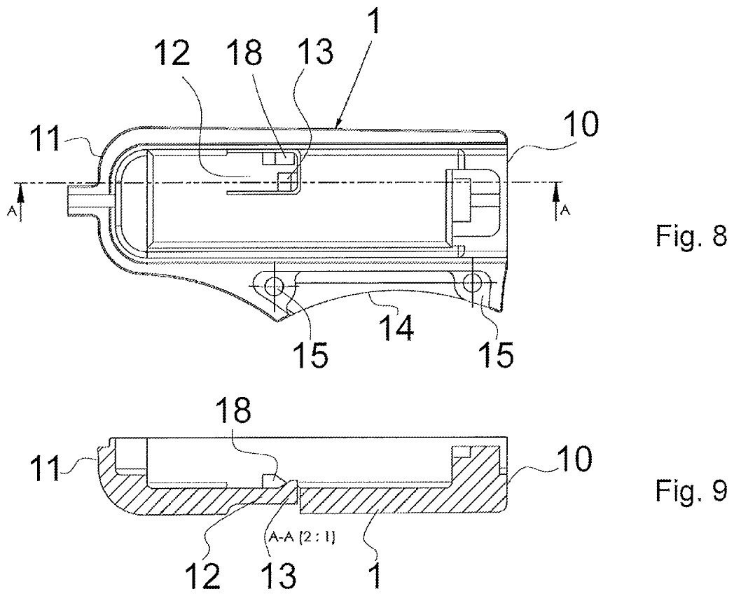

FIG. 8 shows the first housing part 1 from the side lying opposite in comparison to FIG. 6, i.e. looking towards the inner side. The second latching element 12, at the free end of which a latching contour 13 is formed, can in turn be seen. The latching contour 13 interacts with the first latching elements 61 of the outer needle holder 6 in order to form a latching.

In the exemplary embodiments, the first latching element 61 is described as a rigid part on the outer needle holder 6, and the second latching element 12 as an elastically deflectable part. However, the assignment may also be the other way around, i.e. the first latching element 61 can be elastically deflectable, and the second latching element 12 can be arranged rigidly. The first and the second latching elements 12, 61 can each also be elastically deflectable. In the exemplary embodiments, the latching contour 13 is illustrated and described as a latching protrusion. The latching contour 13 may also be configured differently, e.g. as latching depression, latching recess or any other type of latching edge.

Receiving elements 15, in which receiving pins of the finger grip element 8 can be accommodated in order to connect the latter to the housing parts 1, 2 in a form-fitting manner, can also be seen.

In addition, the previously mentioned second latching element 12 with the latching contour 13 can readily be seen in the sectional view of FIG. 9.

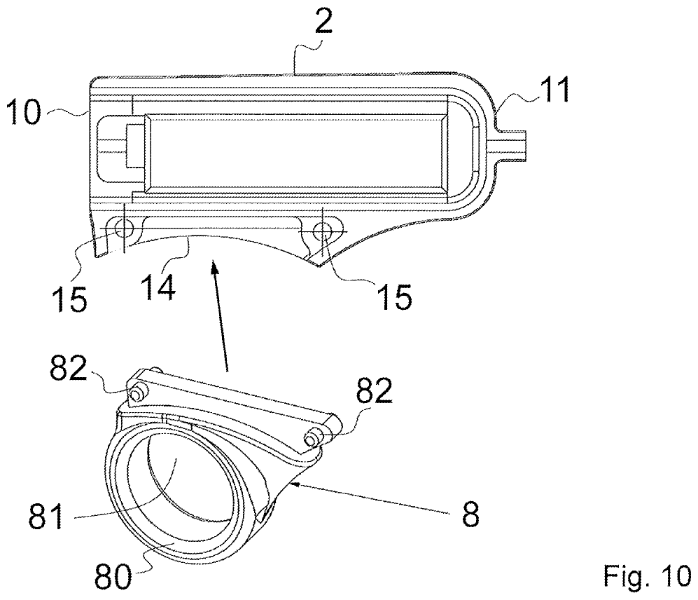

FIG. 10 clarifies the attaching of the finger grip element 8 to the housing parts, here using the example of the housing part 2, which is illustrated in side view. The housing part 2 also has the receiving elements 15. It can be seen that the finger grip element 8 has an annular finger receptacle 80 with an inner recess 81. The inner recess 81 is dimensioned such that only one finger of a user can be placed there. On the side facing the attachment point 14, the finger grip element has receiving pins 82 which are designed for insertion into the receiving elements 15 of the housing parts 1, 2.

The biopsy needle device can be used as follows. First of all, the biopsy needle device is to be pretensioned into one of the pretensioning positions 1 or 2 in accordance with the cutting length markings 54 by pulling out the displacement element 5 at the manual operating element, i.e. at the triggering element 50. In the process, one of the first latching elements 61 latches to the second latching element 12. If the tissue sample is to be extracted within the scope of the biopsy, the triggering element 50 is pressed manually. By this means, the displacement element 5 is already displaced somewhat in the distal direction. In the process, the second latching element 12 is somewhat deflected by the latching release element 55, as a result of which the latching of the latching contour 13 to the first latching element 61 of the outer needle holder 6 is released. Owing to the release of said latching, the outer needle holder 6 is from this time on automatically moved further in the distal direction by the triggering spring 7. In this way, the outer needle 3 is pushed over the region of the tissue window 40 of the inner needle 4. The desired tissue sample is severed by means of the outer needle 3 and stored in that region of the tissue window 40 which then lies within the outer needle 3. The biopsy needle device can then be removed from the patient.

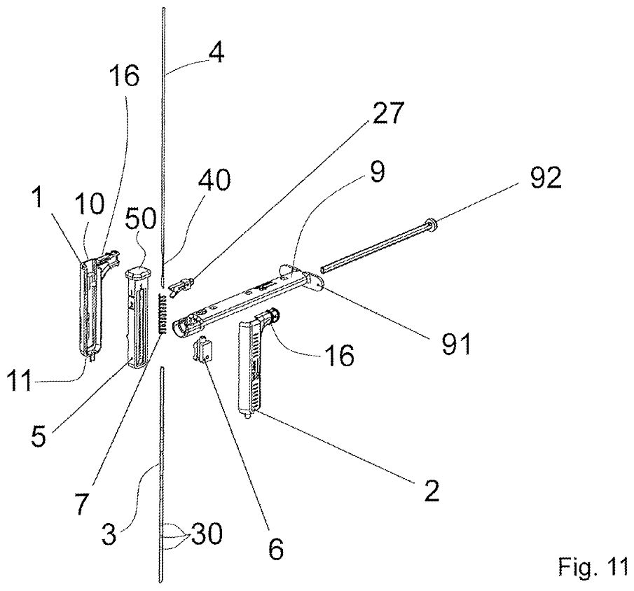

FIG. 11 shows an embodiment of a biopsy needle device in which the housing parts 1, 2, the outer needle 3, the inner needle 4, the displacement element 5, the outer needle holder 6 and the triggering spring 7 can be configured substantially identically to the biopsy needle device described previously with reference to FIGS. 1 to 10. In contrast to the previously described biopsy needle device, further elements are provided in FIG. 11, said elements permitting an external triggering, i.e. triggering at a distance, of the pretensioned biopsy needle device by means of an external actuator 9. For this purpose, the biopsy needle device can be configured either with or without a finger grip element.

In the exemplary embodiment illustrated, a finger grip element is not provided. Instead, a docking holder 16 which is configured for holding the external actuator 9 is integrally formed on the housing 1, 2. The external actuator 9 has fastening elements 90 with which it can be fastened, e.g. in a form-fitting manner, to the docking holder 16. A second triggering element 27 which is mounted displaceably in a direction transversely with respect to the longitudinal axis of the outer needle 3 can be arranged within the docking holder 16. By application of a compressive force at that end of the second triggering element 27 which is remote from the displacement element 5, the displacement element 5 can be moved by means of the second triggering element 27 in the same distal direction as by direct manual pressure actuation of the triggering element 50 of the displacement element 5. So that the second triggering element 27 can be actuated remotely, an actuation plunger 92 is guided through the actuator 9. The actuator plunger 92 can be actuated manually at an end region 91 of the external actuator 9, at which the latter can also be held manually by the user. For the deflection of the movement direction of the second triggering element 27 in the movement direction of the displacement element 5 that is substantially orthogonal thereto, a slope can be integrally formed on the second triggering element 27 in a manner similar to a ramp, which permits this conversion of the movement direction.

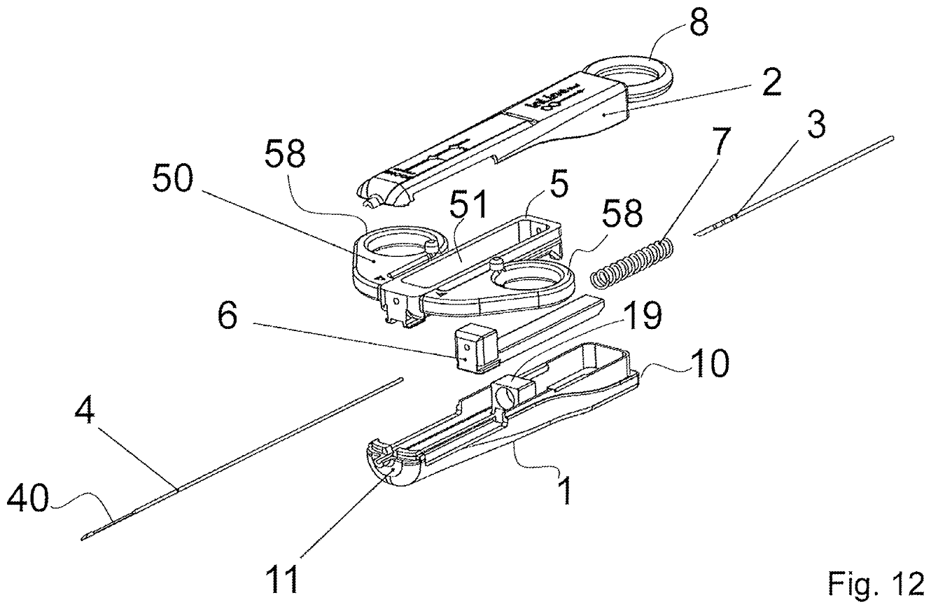

FIG. 12 shows a further embodiment of a biopsy needle device in an exploded illustration. The biopsy needle device has a housing 1, 2 with a first housing part 1 and a second housing part 2. A finger grip element 8 connected immovably to the housing 1, 2 is arranged on the housing 1, 2 at the proximal end 10. The finger grip element 8 can be integrally formed in one piece, e.g., on one or both of the housing parts 1, 2.

The biopsy needle device likewise has a semiautomatic movement mechanism which, with regard to the design and the functioning, substantially corresponds to the previously described movement mechanism of the other embodiments. There is in turn a displacement element 5, an outer needle holder 6 and a triggering spring 7, and of course also the outer needle 3 and the inner needle 4. The outer needle holder 6 serves for holding the outer needle 3 which is fixed movably thereon. The outer needle holder 6 is in turn guided at least with a partial region in an interior space 51 of the displacement element 5. The triggering spring 7 is in turn clamped between the outer needle holder 6 and a spring bearing 19 fixed on the housing.

In contrast to the previously described embodiments, in the case of the embodiment described here the displacement element 5 is manually operable by the user laterally on the housing 1, 2 rather than at the proximal end of the biopsy needle device. For this purpose, one or two finger grip elements 58 is or are integrally formed laterally on a frame-shaped main body of the displacement element 5 or connected thereto. The lateral finger grip elements 58 protrude laterally out of the housing 1, 2 when the components are assembled. By means of this type of construction, the displacement element 5 is moved to and fro only in the region of the longitudinal extent of the housing 1, 2, but does not protrude out of the housing 1, 2 at the proximal end with different dimensions, depending on the pretensioning position, as in the case of the previously described embodiments. In this way, the entire length of the housing region of the biopsy needle device always remains the same during use, irrespective of the tensioning state.

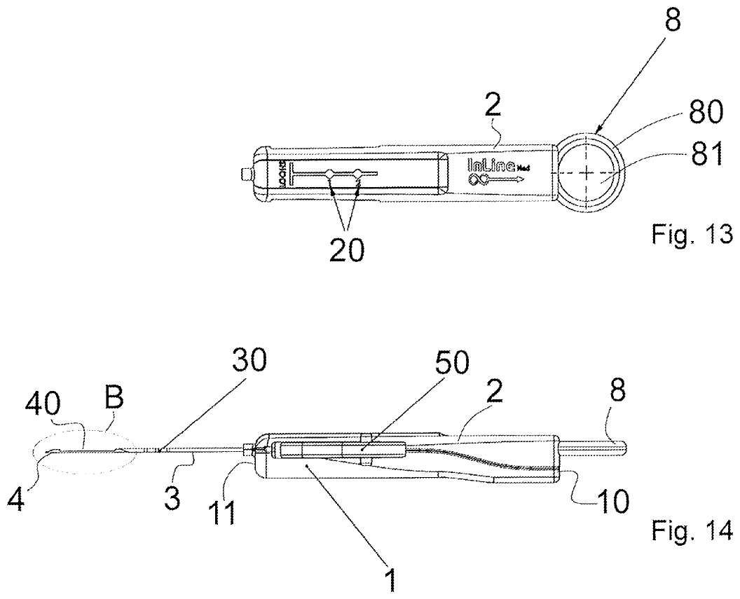

The biopsy needle device explained with reference to FIG. 12 is additionally illustrated in a top view in FIG. 13 and in a side view in FIG. 14. It can be seen in FIG. 13 that different pretensioning positions, which can be read by way of cutting length markings 20, can also be set here. The cutting length markings 20 can be provided, for example, on one of the housing parts 1, 2. The finger grip element 8 in turn has an annular finger receptable 80 with an inner recess 81. The inner recess 81 is dimensioned in such a manner that only one finger of a user finds space therein.

It can be seen with reference to FIG. 14 that the biopsy needle device is constructed with regard to the outer needle 3, the inner needle 4 and in particular the tissue window 40 comparably to the embodiments described with reference to FIGS. 2 and 3.

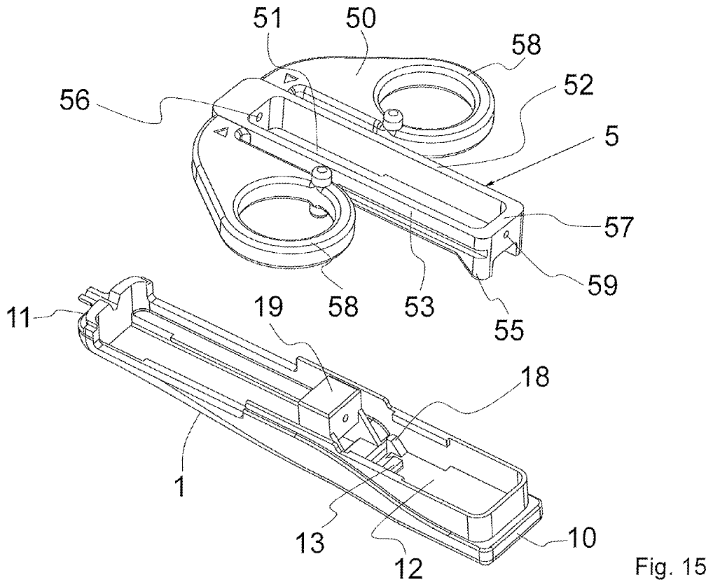

FIG. 15 clarifies the design of the displacement element 5 with its manual operating region with the finger grip elements 58 which, combined, form a triggering element 50 and a pretensioning operating element. The finger grip elements 58 can be configured with regard to the design and dimensioning comparably to the finger grip element 8. The displacement element 5 in turn has a frame-like design with an interior space 51 which forms a receiving space for the displacement element 5. Said interior space 51 is surrounded by longitudinal struts 52, 53 which are connected at the distal end by a transverse strut 57. The transverse strut 57 has an outer needle guide 56 for the passage of the outer needle 3.

In the housing part 1, the spring bearing 19, which is fixed on the housing, for the triggering spring 7 can be seen. The longitudinal struts 52, 53 are guided laterally past the spring bearing 19. In the region between the proximal end 10 and the spring bearing 19, the housing part 1 has a second latching element 12, e.g. an elastically deflectable latching tab on which a latching contour 13, e.g. a latching protrusion, is arranged. The latching contour 13 serves for latching the outer needle holder 6 in the respectively desired latching position. On the displacement element 5, e.g. on one or both longitudinal struts 52, 53, there is a latching release element 55 which comes into contact with a release contour 18 of the second latching element 12 during a triggering operation and, in the process, deflects the second latching element 12 downward and in this way releases the latching of the latching contour 13 to first latching elements 61 of the outer needle holder 6. The latching release element 55 can be located on the displacement element 5 entirely at the rear below and in each case laterally from the outer needle receptable 59.

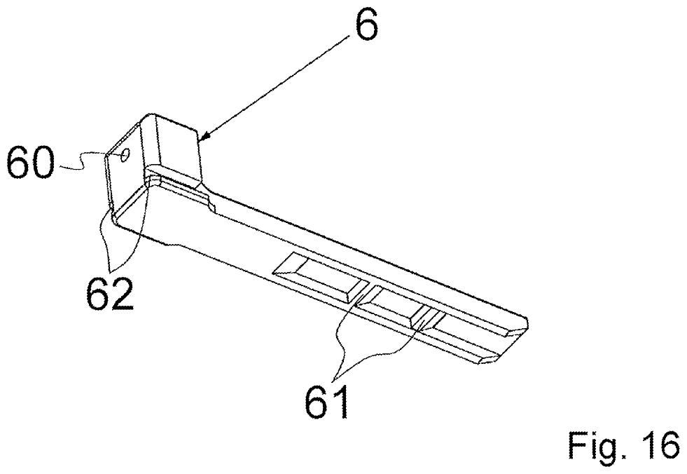

FIG. 16 shows the outer needle holder 6 in a view in which its first latching element 61 can be seen. The latter are formed in this case on the underside of an extended region such that they can come into contact with the latching contour 13 on the housing part 1. For the longitudinal guidance, the outer needle holder 6 has one or two guide elements 62, which guide element or guide elements is/are guided in the longitudinal strut 53 of the displacement element 5.

The function and operation of the biopsy needle device in this embodiment substantially corresponds to the manner of operation already described previously. In contrast thereto, the tensioning operation takes place here via the manual operating region having the finger grip elements 58, i.e. a different manner of gripping and finger support than in the first embodiment is possible. In contrast thereto, the biopsy needle device described here can be gripped with three fingers and in this way operated single handedly, both during the pretensioning and during the triggering movement. The finger grip elements 58 therefore also serve as a triggering element 50 for triggering the movement, which is assisted by the triggering spring, of the outer needle 3 in relation to the inner needle 4.

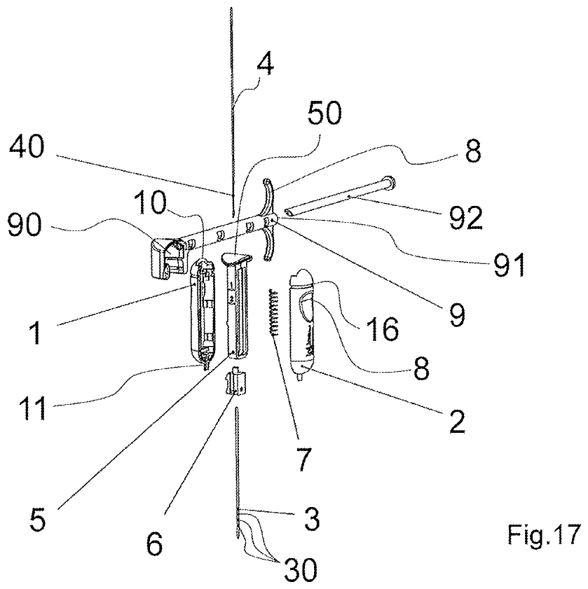

FIG. 17 shows an embodiment of a biopsy needle device and of an external actuator 9 which serves for remotely triggering the triggering element 50 of the biopsy needle device. In contrast, e.g. to the embodiment of FIG. 11, the biopsy needle device according to FIG. 17 does not have a docking holder 16 for docking the external actuator 9. Instead, the external actuator 9 is formed by means of its fastening element 90 in such a manner that it can nevertheless be fastened to the proximal end 10 of the housing 1, 2 of the biopsy needle device, for example, by means of a form fit and/or force fit. In this case, the external actuator 9 with the fastening element 90 can simply be pulled over the proximal end 10 of the biopsy needle device. The biopsy needle device has to be previously pretensioned into a desired pretensioning position. The triggering element 50 is then triggered, similarly as in the embodiment of FIG. 11, by an actuation plunger 92 which is movable in the transverse direction with respect to the longitudinal direction of the outer needle 3 and can be actuated manually (by pressing) at the end of the external actuator. In this embodiment, the actuation plunger 92 acts mechanically directly on the triggering element 50. In contrast to the embodiment of FIG. 11, a second separate triggering element 27 is therefore not required.

Otherwise, the biopsy needle device according to FIG. 17 can be formed comparably to one of the biopsy needle devices as have been described previously.

FIG. 17 illustrates a further characteristic of the biopsy needle device, namely the embodiment of a finger grip element 8 as a notch or indentation arranged laterally on the housing 1, 2. This is also advantageous for ergonomic operability of the biopsy needle device.

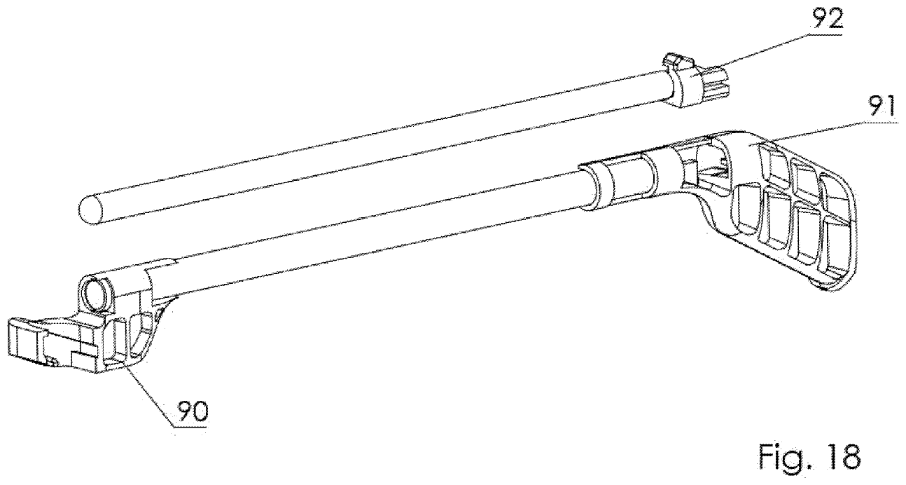

FIG. 18 shows an embodiment of an external actuator 9 in an exploded illustration. The external actuator 9 according to FIG. 18 has a fastening element 90, the external actuator 9 being able to be fastened by a form fit and/or force fit to the proximal end of a housing of a biopsy needle device, as has been described previously. It becomes clear that the external actuator 9 has a pistol grip in its end region 91, with the actuation plunger 92 being undertaken by pushing the external actuator 9 forward. The actuation plunger 91 can be actuated here manually, for example by pressing.

The pistol grip has the advantage that it constitutes an ergonomically adapted handle for the user, and therefore the force which is to be applied can be minimized and at the same time the engagement precision increased. Furthermore, improved grippiness and stability in the guiding and orientation of the biopsy needle device is achieved.

It becomes clear that the pistol grip is arranged laterally on the end region 91 of the external actuator 9. However, it is also conceivable for the pistol grip to be arranged from above or below.

Otherwise, the external actuator 9 according to FIG. 18 can be designed comparably to one of the external actuators 9 as described previously.

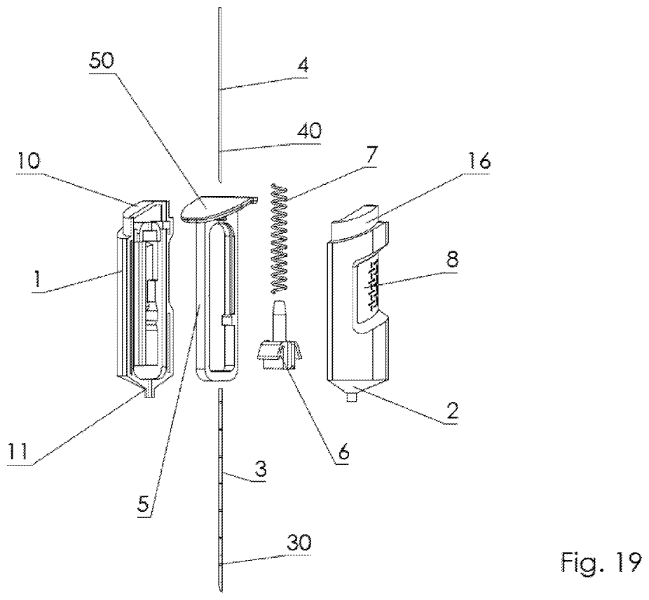

FIG. 19 shows an embodiment of a biopsy needle device in an exploded view. In the case of the biopsy needle device according to FIG. 19, the latching of the outer needle holder 6, in contrast, e.g., to the embodiment of FIG. 1, is released via two second latching elements 12 which are designed to be latchable to first latching elements 61, the first latching elements 61 being arranged in the interior of the housing parts 1, 2. The first latching elements 61 are therefore no longer accessible from the outside, with the housing parts 1, 2 in the mounted state being closed per se.

It becomes clear that two first latching elements 61 protrude in the manner of wings from the outer needle holder 6. The second latching elements 12 can be configured, for example, as elastically deflectable webs, the second latching elements 12 interacting with the first latching elements 61 of the outer needle holder 6 to fix the outer needle holder 6 in the desired latching positions.

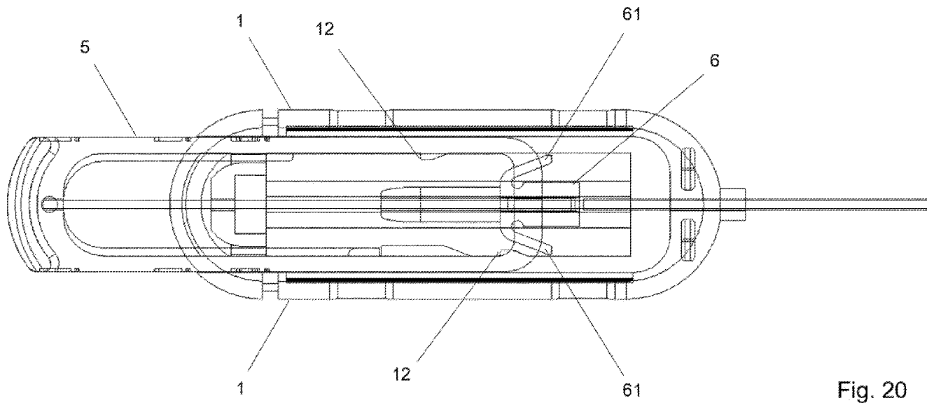

FIG. 20 shows the biopsy needle device according to FIG. 19 in a sectioned side view. It becomes clear that, in the exemplary embodiment illustrated, there are two second latching elements 12 which are arranged on mutually opposite sides of the housing parts 1, 2. Said two second latching elements 12 are arranged here at different positions in the direction of longitudinal extent of the housing parts 1, 2 such that the outer needle holder 6 can be fixed in two positions which differ from each other, by at least one of the first latching elements 61 latching to the corresponding second latching element 12.

However, it is also conceivable for the two second latching elements 12 to be arranged only on one of the housing parts 1, 2 at different positions in the direction of longitudinal extent of the corresponding housing part 1, 2.

It can furthermore be seen that the housing parts 1, 2 in the mounted state are closed per se. that is to say that the second latching elements 12 are no longer accessible from the outside. In this way, external influences on the latching can be reduced or avoided.

Otherwise, the biopsy needle device according to FIGS. 19 and 20 can be configured comparably to one of the biopsy needle devices as has been described previously.

### LIST OF REFERENCE SIGNS

1 Housing part
2 Housing part
3 Outer needle
4 Inner needle
5 Displacement element
6 Outer needle holder
7 Triggering spring
8 Finger grip element
9 External actuator
10 Proximal end of the housing
11 Distal end of the housing
12 Second latching element
13 Latching contour
14 Attachment point
15 Receiving element
16 Docking holder for external actuator
18 Release contour
19 Spring bearing
20 Cutting length marking
27 Second triggering element
30 Penetration depth marking
40 Tissue window
50 Triggering element
51 Interior space
52 Longitudinal strut
53 Longitudinal strut
54 Cutting length marking
55 Latching release element
56 Outer needle guide
57 Transverse strut
58 Finger grip element
59 Inner needle receptable
60 Outer needle receptable
61 First latching element
62 Guide elements
80 Finger receptable
81 Inner recess
82 Receiving pin
90 Fastening element
91 End region
92 Actuation plunger

1. A biopsy needle device having at least one hollow outer needle, an inner needle guided in a longitudinally movable manner in the outer needle, and a housing, wherein the biopsy needle device has at least one manually actuable triggering element for triggering a relative movement between the outer needle and the inner needle, and at least one finger grip element for holding and/or for operating the biopsy needle device by means of at least one finger of a user, wherein the biopsy needle device comprises at least one of the following features:
a) the biopsy needle device has only one finger grip element or the biopsy needle device has one or more finger grip elements which are arranged asymmetrically with respect to a longitudinal axis of the outer needle,
b) the biopsy needle device has at least one finger grip element at the proximal end of the housing,
c) the biopsy needle device has precisely three finger grip elements,
d) at least one finger grip element or an arrangement consisting of at least two finger grip elements is mounted displaceably relative to the housing in the direction of the longitudinal axis of the outer needle.

2. (canceled)

3. The biopsy needle device as claimed in claim 1, wherein at least one finger grip element or an arrangement consisting of at least two finger grip elements is configured simultaneously as a triggering element.

4. The biopsy needle device as claimed in claim 1, wherein the biopsy needle device has at least one pretensioning operating element which can be used to carry out a relative movement between the outer needle and the inner needle in a tensioning direction counter to the force of a triggering spring of the biopsy needle device.

5. The biopsy needle device as claimed in claim 4, wherein the pretensioning operating element is configured simultaneously as a triggering element for triggering a relative movement between the outer needle and the inner needle, said relative movement being brought about by the force of the pretensioned triggering spring and being opposed to the tensioning direction.

6. The biopsy needle device as claimed in claim 4, wherein the biopsy needle device has, in addition to the pretensioning operating element, a triggering element for triggering a relative movement between the outer needle and the inner needle, said relative movement being brought about by the force of the pretensioned triggering spring and being opposed to the tensioning direction.

7. The biopsy needle device as claimed in claim 4, wherein the biopsy needle device has a holding mechanism which is used, when the triggering spring is pretensioned, to hold the pretensioning operating element, or a component connected thereto, in at least one holding position, in particular in at least two different holding positions, in relation to the force of the pretensioned triggering spring.

8. The biopsy needle device as claimed in claim 1, wherein one finger grip element, a plurality of finger grip elements or all of the finger grip elements has/have a ring shape, the ring shape being able to be configured as a ring completely closed circumferentially or a ring which is open at one point.

9. The biopsy needle device as claimed in claim 1, wherein one finger grip element, a plurality of finger grip elements or all of the finger grip elements is/are configured as a separate component which is coupled rigidly or movably to the housing.

10. The biopsy needle device as claimed in claim 1, wherein the just single finger grip element of the biopsy needle device is arranged on the housing in a manner laterally offset with respect to the longitudinal axis of the outer needle.

11. The biopsy needle device as claimed in claim 1, wherein the distance between the finger grip element arranged at the proximal end of the housing and the distal end of the housing is unchangeable.

12. The biopsy needle device as claimed in claim 1, wherein the distance between the finger grip element arranged at the proximal end of the housing and other finger grip elements of the biopsy needle device is at least 2 cm or at least 50% of the housing length of the biopsy needle device in every operating state of the biopsy needle device.

13. The biopsy needle device as claimed in claim 1, wherein a docking holder is arranged on the housing of the biopsy needle device for holding an external actuator with which the triggering element can be actuated by the user by means of remote triggering.

14. A set consisting of
a biopsy needle device having at least one hollow outer needle, an inner needle guided in a longitudinally movable manner in the outer needle, and a housing, wherein the biopsy needle device has at least one manually actuable triggering element for triggering a relative movement between the outer needle and a biopsy needle device as claimed in claim 1, and
an external actuator with which the triggering element of the biopsy needle device can be actuated by the user by means of remote triggering.

15. The set as claimed in claim 14, wherein the external actuator has at least one fastening element with which the external actuator is fastenable to the biopsy needle device in a form-fitting and/or force-fitting manner.

16. The set as claimed in claim 14, wherein the external actuator has an actuation plunger for remote actuation of the triggering element of the biopsy needle device, the actuation plunger being longitudinally movable in a direction which runs transversely with respect to the longitudinal axis of the outer needle.

17. A biopsy needle device having at least one hollow outer needle, an inner needle guided in a longitudinally movable manner in the outer needle, and a housing, wherein the biopsy needle device has at least one manually actuable triggering element for triggering a relative movement between the outer needle and the inner needle, wherein the manually actuable triggering element is actuable in an actuating direction which is arranged transversely with respect to the longitudinal axis of the outer needle.

18. The biopsy needle device as claimed in claim 17, wherein at least one finger grip element or an arrangement consisting of at least two finger grip elements is configured simultaneously as a triggering element.

19. The biopsy needle device as claimed in claim 17, wherein the biopsy needle device has at least one pretensioning operating element which can be used to carry out a relative movement between the outer needle and the inner needle in a tensioning direction counter to the force of a triggering spring of the biopsy needle device.

20. The biopsy needle device as claimed in claim 19, wherein the pretensioning operating element is configured simultaneously as a triggering element for triggering a relative movement between the outer needle and the inner needle, said relative movement being brought about by the force of the pretensioned triggering spring and being opposed to the tensioning direction.

21. The biopsy needle device as claimed in claim 19, wherein the biopsy needle device has, in addition to the pretensioning operating element, a triggering element for triggering a relative movement between the outer needle and the inner needle, said relative movement being brought about by the force of the pretensioned triggering spring and being opposed to the tensioning direction.
